(19) United States
(12) Patent Application Publication
(10) Pub. No.: US 2025/0208006 A1
(43) Pub. Date: Jun. 26, 2025

(54) SAMPLE COLLECTION DEVICE

(71) Applicant: Siro Diagnostics, Inc., San Diego, CA (US)

(72) Inventors: Joseph D. Kowalski, San Diego, CA (US); Robert K. Shanahan, San Diego, CA (US); Michael Yep, San Diego, CA (US); Clint Sever, San Diego, CA (US)

(21) Appl. No.: 18/846,226

(22) PCT Filed: Mar. 14, 2023
(86) PCT No.: PCT/US2023/015216

§ 371 Date: Sep. 11, 2024

Related U.S. Application Data
(60) Provisional application No. 63/320,049, filed on Mar. 15, 2022.

(51) Int. Cl. G01N 1/02; A61B 10/02

(52) U.S. Cl. CPC G01N 1/02; A61B 10/02; A61B 2010/0216; G01N 2001/028

(57) ABSTRACT

A sample collection device for collecting a biological sample for analysis in a detection system includes a body including a wand portion, and a tip extending from a proximal end of the wand body and configured to receive a sample. The tip includes a tip body comprising a flexible material, a layer of flocking coupled to the tip body and an enlarged portion that defines convex lateral sides of the tip. The body may extend along an axis and the tip may be configured to flex about the axis.

### CROSS REFERENCE TO RELATED APPLICATION

This application claims the benefit of and priority to U.S. Provisional Patent Application No. 63/320,049, filed Mar. 15, 2022, the contents of which is hereby incorporated herein by reference in its entirety.

### TECHNOLOGICAL FIELD

The present technology relates generally to sample collection devices. In particular, the technology relates to systems and methods for collecting sample for uses in medical testing.

### BACKGROUND

Sample collection devices may be configured to collect cells and other biological material from various regions or locations including, for example, an inner cheek, the throat, a nasal passageway, an ear, the vaginal wall, from urine, from blood, from plasma, from saliva, or from another body part.

### SUMMARY

A set of embodiments relate to a sample collection device for collecting a biological sample for analysis in a detection system includes a body including a wand portion, and a tip extending from a proximal end of the wand body and configured to receive a sample. The tip includes a tip body comprising a flexible material.

In various embodiments, the tip may include layer of flocking coupled to the tip body. According to various embodiments, the tip includes an enlarged portion that defines convex lateral sides of the tip. According to various embodiments, the flexible material includes an elastomeric material. The flocking may include a polyester bicomponent fiber. The body may extend along an axis and the tip is may be configured to flex about the axis. The tip may define a first diameter at a first end proximate the wand portion and a second diameter at a second end opposite the first end, the second diameter being larger than the first diameter. The flocking may be coupled to the tip via adhesive. The flocking, may be configured to absorb the biological sample. The tip body may configured to flex proximate a first inflection point proximate the wand portion. The tip body may be configured to flex proximate a second inflection point proximate between a first end of the tip and a second end of the tip.

Another set of embodiments relate to a sample collection device for collecting a biological sample for analysis in a detection system, comprising a tip extending along an axis, configured to receive a sample. The tip includes a tip body including a flexible material and a layer of flocking coupled to the tip body.

The tip may include an enlarged portion that defines convex lateral sides of the tip. The tip body may include an elastomeric material. The flocking may include a polyester bicomponent fiber. The flocking may be coupled to the tip body via an adhesive. A wand portion may be coupled to to the tip, wherein the tip defines a first diameter at a first end proximate the wand portion and a second diameter at a second end opposite the first end, the second diameter being larger than the first diameter.

### BRIEF DESCRIPTION OF THE DRAWINGS

Exemplary embodiments are described below with reference to the accompanying drawings, wherein like numerals denote like elements. In the drawings:

FIGS. 1A-1B provide schematic depictions of an exemplary analyte detection system for analyzing the presence, absence, and/or quantity of one or more target analytes within a collected sample wherein FIG. 1A shows components uncoupled and FIG. 1B shows components coupled for analysis and charging.

FIG. 1C provides a schematic depiction of another exemplary analyte detection system for analyzing the presence, absence, and/or quantity of one or more target analytes within a collected sample, wherein a charger is not provided.

FIGS. 2A and 2B illustrate perspective views of a conventional exemplary sample collection device which is usable in the analyte detection systems of FIGS. 1A-1C.

FIG. 3 illustrates a perspective view of a sample collection device, according to an example embodiment, which is usable in the analyte detection systems of FIGS. 1A-1C.

FIG. 4A illustrates a side view of the sample collection device of FIG. 3 in an extended orientation.

FIG. 4B illustrates a side view of the sample collection device of FIG. 3 in a collapsed orientation.

FIG. 5 illustrates a perspective view of a wand portion of the sample collection device of FIG. 3.

FIG. 6 illustrates a perspective view of an extension portion of the sample collection device of FIG. 3.

FIG. 7 illustrates a cross sectional view of the sample collection device of FIG. 3 in an extended orientation.

FIG. 8 illustrates a cross sectional view of a distal end the sample collection device of FIG. 3 when the sample collection device is in a collapsed orientation.

FIG. 9 illustrates a perspective view of a locating pin of the sample collection device of FIG. 3.

FIG. 10 illustrates another perspective view of the locating pin of FIG. 9.

FIG. 11 illustrates a partial perspective view of the wand portion of FIG. 5.

FIG. 12 illustrates a partial view of a wand portion, according to another example embodiment.

FIG. 13 illustrates a side view of a portion of a sample collection device including one example tip.

FIG. 14 illustrates a side view of a portion of a sample collection device including another example tip.

FIG. 15 illustrates a side view of a portion of a sample collection device including yet another example tip.

FIG. 16 illustrates a side view of a portion of a sample collection device including still another example tip.

### DETAILED DESCRIPTION OF CERTAIN EMBODIMENTS

In the following detailed description, reference is made to the accompanying drawings, which form part of the present disclosure. The embodiments described in the drawings and description are intended to be exemplary and not limiting. As used herein, the term “exemplary” means “serving as an example or illustration” and should not necessarily be construed as preferred or advantageous over other embodiments. Other embodiments may be utilized and modifications may be made without departing from the spirit or the scope of the subject matter presented herein. Aspects of the disclosure, as described and illustrated herein, can be arranged, combined, and designed in a variety of different configurations, all of which are explicitly contemplated and form part of this disclosure.

One aspect of the disclosure is directed to a system for detecting molecules. In various embodiments, the system includes a cartridge device, a reader device removably coupled to the cartridge device, and a sample collection device. According to various embodiments, the sample device is configured to be exposed to a sample for an analysis, such that the sample collection device receives a sample for medical testing. The sample collection device is further configured to be inserted into the cartridge device, which may be inserted into the reader device. The cartridge device and/or the reader device may cause medical testing to be performed on a portion of the sample collected by the sample collection device.

The sample collection device of various embodiments is configured to collect a sample from a specimen. Sample collection devices may be configured to collect cells and other biological material from any desired region or location, for example, an inner cheek, the throat, a nasal passageway, an ear, from urine, from blood, from plasma, from saliva, or from another body part. One exemplary sample collection device includes a unit that wicks a small droplet of blood or urine into a small capillary channel. In other embodiments, the sample collection device may be configured to collect biological material, particulates, or other chemicals from the environment, such as, for example, from the air or the water, or from a physical surface or other structure.

The sample collection device of various embodiments is sized and shaped to collect a sufficiently large sample from an appropriate location of a specimen such that it is possible, using the other devices described below, to detect the presence, absence, and/or quantity of one or more target analytes in and/or on the specimen. For example, for some target analytes, such as ones associated with a virus causing cold or flu-like symptoms, the sample collection device may be a nasal swab; the swab is sized and shaped to collect a sufficient amount of sample from a nasal passageway of an individual to enable detection of target analytes associated with the virus causing cold or flu-like symptoms, if present in the individual. For other target analytes, such as, for example, ones associated with strep throat, the sample collection device may be a throat swab shaped to collect (e.g., scrape, absorb, etc.) sufficient cells from an individual's throat or mouth. As another example, the sample collection device appropriate for collecting a target analyte associated with HIV may include a blood lancet. In another example, a sample collection device configured to collect urine may be appropriate for collecting target analytes for various tests, including, for example, tests for tracking fertility, pregnancy, drug levels, sexual health markers, and/or other various analytes. A sample collection device for collecting fluid, such as urine, blood, plasma, or saliva, may include features for compressing a wicking portion of the device to expel sample absorbed on the wicking portion for analyzing the expelled sample. In yet a further aspect, the sample collection device is shaped to collect a sample from a solid surface, e.g., on a medical device, on medical equipment or from the surface of a food preparation surface such as a cutting board or flat surface.

The sample collection device of various embodiments is configured to collect a sample from a specimen. Sample collection devices may be configured to collect cells and other biological material from any desired region or location, for example, an inner cheek, the throat, a nasal passageway, an ear, from urine, from blood, from plasma, from saliva, or from another body part. One exemplary sample collection device includes a unit that wicks a small droplet of blood or urine into a small capillary channel. In other embodiments, the sample collection device may be configured to collect biological material, particulates, or other chemicals from the environment, such as, for example, from the air or the water, or from a physical surface or other structure.

In various embodiments, a cartridge is formed of a housing, which defines an enclosed space and has various features that enable the cartridge to do one or more of the following: receive a sample with target analytes from a sample collection device, store the sample with sample preparation reagents, provide a space for mixing and binding of the target analytes with sample preparation reagents, provide an analysis zone wherein bound target analytes localize over sensors for detection, provide a fluid medium for transporting the bound target analytes to the analysis zone, store and provide a substrate that can undergo a detectable reaction when introduced to the bound target analytes, provide a fluid medium for transporting the substrate to the bound target analytes in the analysis zone, and provide a waste collection zone where waste is stored.

In various embodiments, the cartridge is a substantially closed system wherein the reactions needed to detect the presence, absence, and/or quantity of one or more target analytes occur within the cartridge. The cartridge of such embodiments is said to be “substantially closed” because the only inputs needed into the cartridge system are one or more of the following: a sample from a specimen, energy to facilitate mixing and binding, and a magnetic force to facilitate localization of bound target analytes within an analysis zone; the only outputs from the cartridge are electrical signals. In various embodiments, the cartridge is target-analyte-specific with the included sample preparation reagents selected to detect one or more specific target analytes. Different cartridge types include different reagents intended to identify different target analytes. For example, different cartridge types may include inflammation, influenza, testosterone, fertility, HIV, and Vitamin D which each include application-specific reagents intended to identify different target analytes.

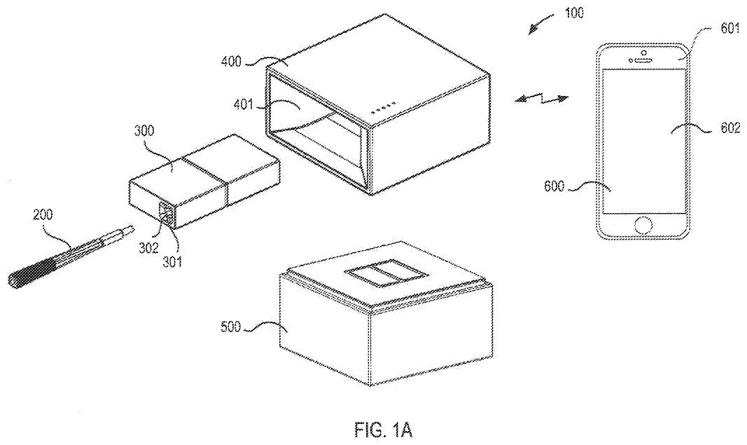

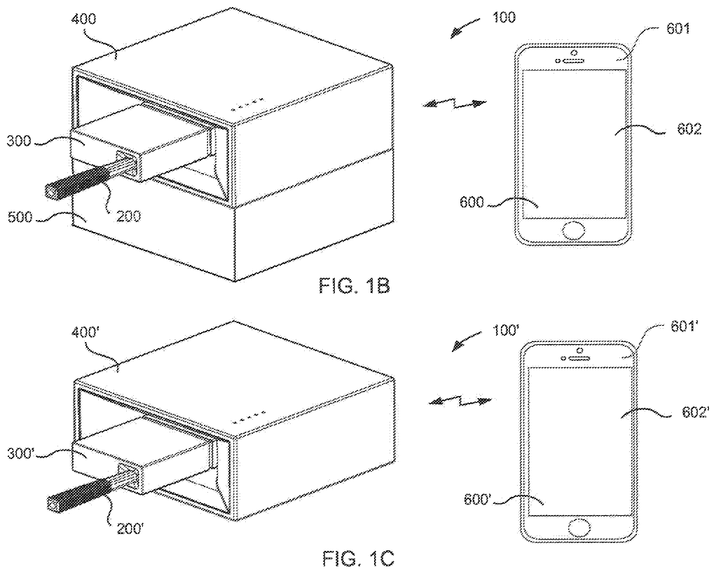

Referring now to FIGS. 1A and 1B, an exemplary analyte detection system is shown, according to an example embodiment. The detection system 100 may include a sample collection device 200, a cartridge device 300, a reader device 400, a charger 500, and/or a software-based detection interface system 600. The detection system 100 may be used to detect the presence, absence, and/or quantity of one or more target analytes.

The sample collection device 200 is configured to be exposed to a sample for analysis. For example, the sample collection device 200 may be exposed to a biological sample, such as, but not limited to, blood, plasma, urine, saliva, mucous, cellular material and/or other biological material for determining the presence, absence, and/or quantity of one or more target analytes within the sample. In addition or alternatively, the sample collection device is exposed to a solid or other surface that is suspected of harboring a target analyte, e.g., a food-borne pathogen and the surface is a cooking or food preparation surface.

The cartridge device 300 is configured to analyze the sample collected with the sample collection device 200. The cartridge device 300 may include an input tunnel 301 that extends from an aperture 302 into the cartridge housing. The input tunnel 301 is configured to permit insertion of the sample collection device 200 as shown in FIG. 1B such that the collected sample may be analyzed within the cartridge device 300. Based on the analysis, the cartridge device 300 is configured to generate electric signals indicative of the presence, absence, and/or quantity of one or more target analytes within the sample.

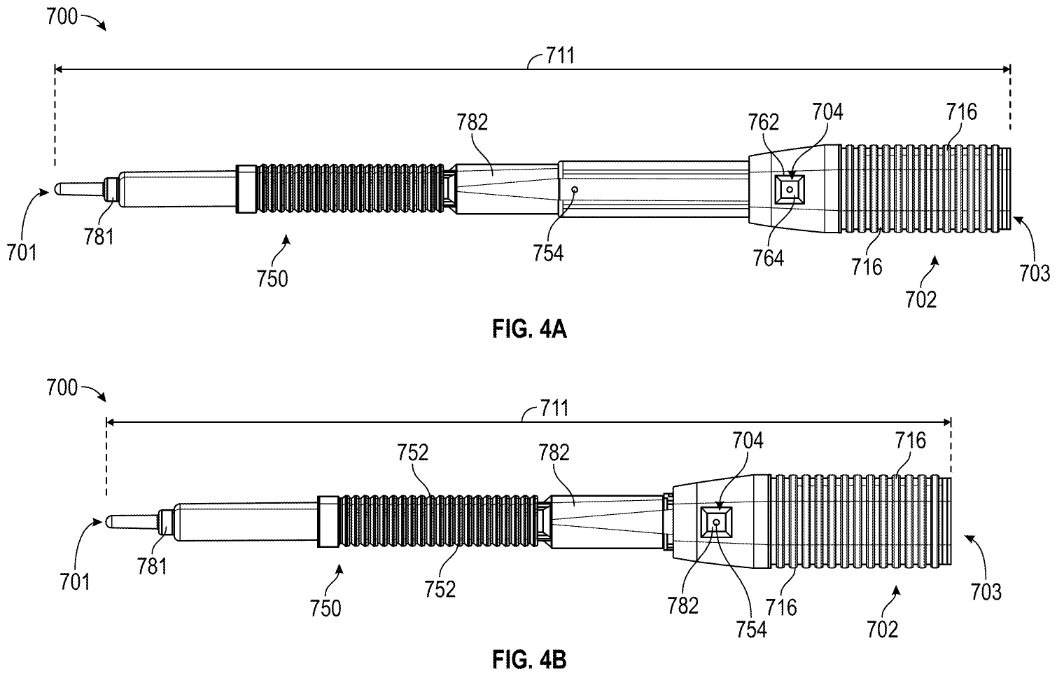

The reader 400 is configured for electric coupling with the cartridge device 300 to permit transmission of the electric signals indicative of the presence, absence, and/or quantity of one or more target analytes within the sample generated by cartridge device 300. Cartridge device 300 may be electrically coupled to reader 400 by inserting the cartridge device 300 within reader opening 401 of reader 400 as shown in FIG. 1B such that respective electrical connectors of cartridge device 300 and reader 400 contact one another. Reader 400 may include a computer readable medium with instructions that, when executed by a processor of reader 400, cause electrical components of the cartridge device 300 to perform steps for analyzing the sample on the sample collection device 200. According to various embodiments, the instructions are not executed until the cartridge device 300 is electrically coupled to the reader 400 and the sample collection device 200 is suitably disposed within the cartridge device 300, for example, as shown in FIG. 1B or 4A.

In one embodiment, the sample collection device 200 and the cartridge device 300 are each disposable and designed for one time use while the reader 400 is designed for multi-use and for receiving many different cartridge devices throughout the life of the reader 400 such that many samples are analyzed by the reader 400 for determining the presence, absence, and/or quantity of one or more target analytes within the respective samples. Such a configuration is expected to promote sanitary use of the system, as the components exposed to the sample are disposable, while reducing costs as the components with more expensive electronics, e.g., the reader 400, may be used repeatedly.

The charger 500 is configured to charge one or more batteries within the reader 400, e.g., via respective inductive coils disposed within the housings of the charger 500 and the reader 400. The charger 500 may be plugged into a conventional socket, e.g., via a cord or a cord with an AC to DC power converter, for charging components within the charger 500 to permit charging of the reader 400.

As will be readily apparent to one skilled in the art, the detection system need not require a charger. For example, referring to FIG. 1C, the detection system 100′ is constructed similarly to the detection system 100 of FIGS. 1A and 1B, wherein like components are identified by like-primed reference numbers. Thus, for example, the cartridge device 300′ in FIG. 1C corresponds to the cartridge device 300 of FIGS. 1A and 1B, etc. As will be observed by comparing FIGS. 1C and 1B, the detection system 100′ does not include the charger 500. In such an embodiment, the reader 400′ may be plugged into a conventional socket, e.g., via a cord or a cord with an AC to DC power converter, for powering components of the reader 400′ and/or the reader 400′ may include a suitable battery such as a replaceable battery or rechargeable battery and the reader 400′ may include circuitry for charging the rechargeable battery, and a detachable power cord.

In FIGS. 1A and 1B, software-based detection interface system 600 is installed and runs on the computing device 601 to permit a user to review analyte detection test results, e.g., on a display 602 of the computing device 601, according to various example embodiments. The computing device 601 may be, for example, a smartphone, smartwatch, tablet, wearable device, a laptop or other computer. As shown in FIGS. 1A and 1B, the reader 400 may communicate with the computing device 601 wirelessly to transmit data indicative of the presence, absence, and/or quantity of one or more target analytes based on the electrical signals generated within the cartridge device 300. In addition or alternatively, a removable wired connection, such as a cable connection, may be provided between the reader 400 and the computing device 601. The software-based detection interface system 600 may include a computer readable medium with instructions that, when executed by a processor of the computing device 601, cause the display 602 to display information indicative of the presence, absence, and/or quantity of one or more target analytes. It should be appreciated that, according to various embodiments, the software-based detection interface system 600 may be omitted from the detection system 100. For example, the reader device 400 may include a display or other visual indication system that provides results and/or other information to a user of the detection system 100 without the need for an external computing device 601 and/or a software-based detection interface system 600.

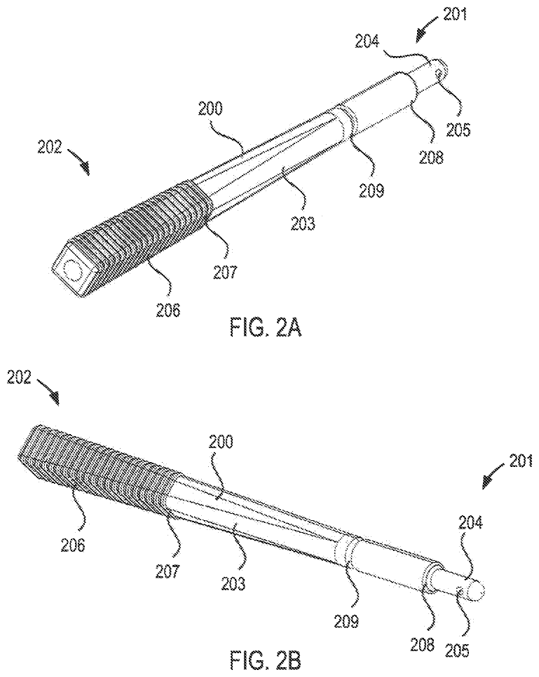

Referring now to FIGS. 2A and 2B, a conventional sample collection device 200 is illustrated, according to an example embodiment. The sample collection device 200 is configured to collect a small quantity of a sample to be analyzed and configured for full or partial insertion within the cartridge device 300 after sample collection. The sample collection device 200 may include a distal portion 201, a proximal portion 202, and a shaft 203 extending therebetween. The distal portion 201 may include a tip 204 having a tube 205 therein. The sample collection device 200 also may include a handle 206, a proximal sealing zone 207, a distal sealing zone 208, and/or an engagement zone 209.

The distal portion 201, including the tip 204, is configured to be exposed to a sample such that, at most, a predetermined volume of the sample is disposed in the tube 205 for analysis. Collection of a predetermined volume of the sample is expected to promote accuracy of analyte analysis as a substantially predetermined quantity of the sample will be analyzed. The tip 204 may include a transparent portion to permit a collector to verify that sample is disposed in the tube 205. For example, the collector may view sample located within the tip 204 view the transparent portion. The tip 204 may have a rounded end as illustrated although various shapes may be used including any blunt or substantially blunt tip shape. The tip 204 may be configured to collect a sample from any desired region or location, for example, an inner cheek, the throat, the mouth, a nasal passageway, an ear, from urine, from blood, from plasma, from saliva, or from another body part.

The proximal portion 202 may include a handle 206 sized and shaped to be held by a collector's hand. The handle 206 may include gripping protrusions as illustrated. The handle 206 may further the lock the sample collection device 200 within the input tunnel of cartridge device 300. The sample collection device 200 also may include a proximal sealing zone 207 configured for sealing the input tunnel of the cartridge device 300 when the sample collection device 200 is inserted in the input tunnel. The proximal sealing zone 207 may include one or more protrusions extending around the shaft 203 and sized greater than the input tunnel opening so as to seal off the input tunnel. As such, the one or more protrusions may further lock sample collection device within the input tunnel of the cartridge device 300. The handle 206 may be breakable or otherwise removable from the remainder of the sample collection device 200 following insertion of the remainder of the sample collection device 200 into the cartridge device 300.

The shaft 203 is elongated to facilitate easy and sanitary collection, with a collector's hand being distanced from the site of collection. For example, the shaft 203 may be elongated such that the tip 204 may be exposed to a sample within an inner cheek, the throat, the mouth, a nasal passageway, an ear, from urine, from blood, from plasma, from saliva, etc. to collect fluid, cells, and other biological material, while the handle 206 is not exposed to the sample. The shaft 203, the tip 204, and the handle 206 may be formed of the same material or of different materials. The shaft 203, the tip 204, and the handle 206 may be formed of a plastic. The sample collection device 200 may be pre-packaged within sterile packaging and may be configured for one-time use.

The sample collection device 200 may have a distal sealing zone 208 for facilitating the formation of a liquid-tight seal between the sample collection device 200 and the cartridge device 300 after insertion of the sample collection device 200 into the cartridge device 300. For example, the distal sealing zone 208 may be sized and shaped to seal the collected sample on the tip 204 and fluid within a sample preparation reservoir of the cartridge device 300 within the cartridge device 300. The distal sealing zone 208 may be of greater radial size than the tip 204. For example, the distal sealing zone 208 may include a shoulder extending further from the longitudinal axis of the sample collection device 200 than the tip 204 such that shoulder abuts against a portion of the cartridge device 300, e.g., a shuttle, to form the liquid-tight seal. In this manner, the sample may be sealed within the cartridge device 300 to reduce leakage and exposure of the sample outside the cartridge. In addition, the shoulder may be used to move a seal piercer to vent one or more reservoirs within the cartridge device 300 before, during, or after formation of the liquid-tight seal.

The sample collection device 200 may include an engagement zone 209 configured for engagement with one or more components of the cartridge device 300. For example, the engagement zone 209 may be configured to be coupled, permanently or temporarily, to a seal piercer of the cartridge device to move the seal piercer within the cartridge device responsive to movement of the sample collection device 200. The engagement zone 209 also may facilitate fixed engagement between the sample collection device 200 and the cartridge device such that the sample collection device 200 is mated irreversibly and immovably with the cartridge when the sample collection device 200 is inserted a predetermined distance in the input tunnel of the cartridge. The engagement zone 209 may be a groove around the shaft 203 as illustrated or may be multiple grooves extending a shorter distance from the longitudinal axis than the regular shaft surface and/or may be one or more protrusions extending a greater distance from the longitudinal axis of the sample collection device 200.

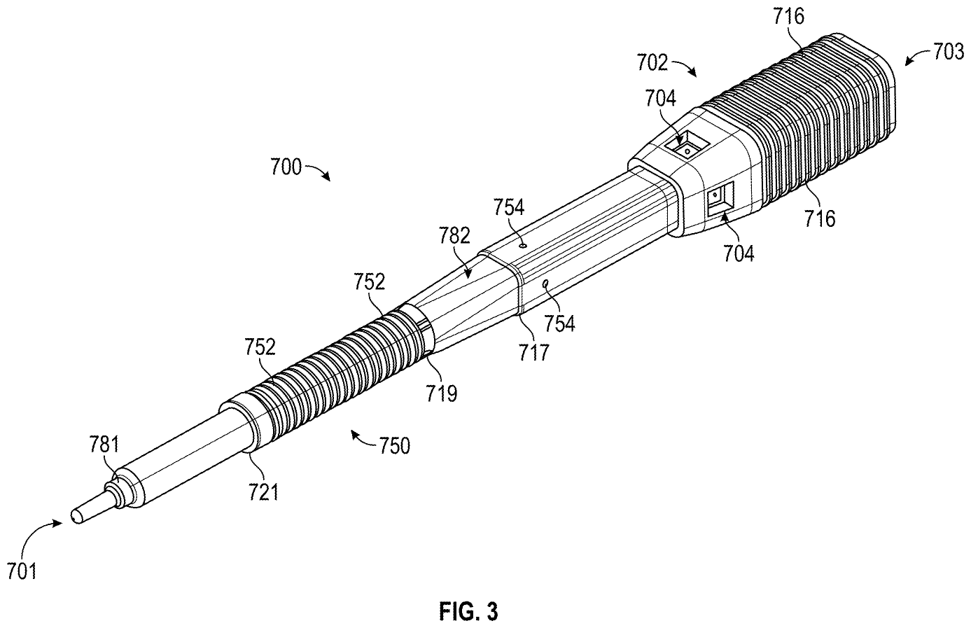

Referring now to FIGS. 3-4B, a sample collection device 700 is shown, according to an example embodiment. The sample collection device 700 is configured to be exposed to a sample such that some or all of the sample may be collected for analysis. For example, the sample collection device 700 may be exposed to a biological sample, such as, but not limited to, blood, plasma, urine, saliva, mucous, cellular material and/or other biological material for determining the presence, absence, and/or quantity of one or more target analytes within the sample. In addition or alternatively, the sample collection device 700 is exposed to a solid or other surface that is suspected of harboring a target analyte, e.g., a food-borne pathogen and the surface is a cooking or food preparation surface. The sample collection device 700 may share one or more features with any of the sample collection devices described herein (e.g., the sample collection device 200). Further, the sample collection device 700 may be used in combination with one or more of the components described herein (e.g., the cartridge device 300, the reader device 400, the charger 500, and/or the software-based detection interface system 600, etc.).

The sample collection device 700 is configured to collect a sample to be analyzed and configured for full or partial insertion within the cartridge device 300 after sample collection. The sample collection device 700 extends from a tip 701 (e.g., a first end) to a second end 703, the sample collection device 700 including a shaft 782 positioned therebetween. The sample collection device 700 may also include a first handle that is defined by a first plurality of ridges 752 and a second handle that is defined by a second plurality of ridges 716.

The tip 701 is configured to be exposed to a sample such that a volume of sample may be collected. According to various embodiments, the tip 701 may be covered with a soft and/or absorbent material. For example, a layer of flocking may be applied to the tip 701. The flocking may be a polyester bicomponent fiber that is attached to the tip 701 with an adhesive. According to various embodiments, the flocking may provide an absorbent layer to collect and retain the target sample. According to various embodiments, the flocking may function to cushion the tip 701 and reduce irritation or damage to sensitive tissues during use of the sample collection device 700.

Additionally or alternatively, the tip 701 may be configured to be exposed to a sample such that, at most, a predetermined volume of the sample is disposed in a tube located within the sample collection device 700. Collection of a predetermined volume of the sample is expected to promote accuracy of analyte analysis as a substantially known quantity of the sample will be analyzed. The tip 701 may be transparent to permit a collector to verify that sample is disposed in the tube. The tip 701 may have a rounded end as illustrated although various shapes may be used including any blunt or substantially blunt tip shape. The tip 701 may be configured to collect a sample from any desired region or location, for example, a vaginal wall, an inner cheek, the throat, the mouth, a nasal passageway, an ear, from urine, from blood, from plasma, from saliva, or from another body part.

As discussed above, the sample collection device 700 may include a second handle defined by a second plurality of ridges 716. The second handle may further lock the sample collection device 700 within the input tunnel of cartridge device 300 in a similar fashion as described above with respect to the sample collection device 200. Additionally or alternatively, the sample collection device 700 may include a proximal sealing zone 717 configured to seal the input tunnel of the cartridge device 300 when the sample collection device 700 is inserted in the input tunnel in a similar fashion as the proximal sealing zone 207 described above. The sample collection device 700 further includes an engagement zone 719 configured for engagement with one or more components of the cartridge device 300. For example, the engagement zone 719 may function in a similar manner as the engagement zone 209 described above. The sample collection device 700 further includes a distal engagement zone 721. The distal engagement zone 721 is configured for engagement with one or more components of the cartridge device 300. According to various embodiments, the engagement zone 721 functions in a similar manner as the engagement zone 209 described above. As shown, the sample collection device 700 further includes a distal sealing zone 781 for facilitating the formation of a seal (e.g., a liquid tight seal, an air tight seal, etc.) between the sample collection device 700 and the cartridge device 300 after insertion of the sample collection device 700 into the cartridge device 300. For example, the distal sealing zone 781 may function in a similar manner as the distal sealing zone 208 described above.

As shown, the sample collection device 700 includes a body defined by an extension portion 702 and a wand portion 750 coupled to the extension portion 702. According to various embodiments, the extension portion 702 and the wand portion 750 are configured to translate relative to one another to alter a length 711 of the sample collection device 700. As shown, the length 711 is defined be the distance between the tip 701 and the second end 703 of the sample collection device 700. For example, the sample collection device 700 may be convertible from at least a collapsed orientation (e.g., shown in FIG. 4b) to an extended orientation (e.g., shown in FIG. 4a). According to various embodiments, the sample collection device 700 may further be convertible from at least the extended orientation to the collapsed orientation.

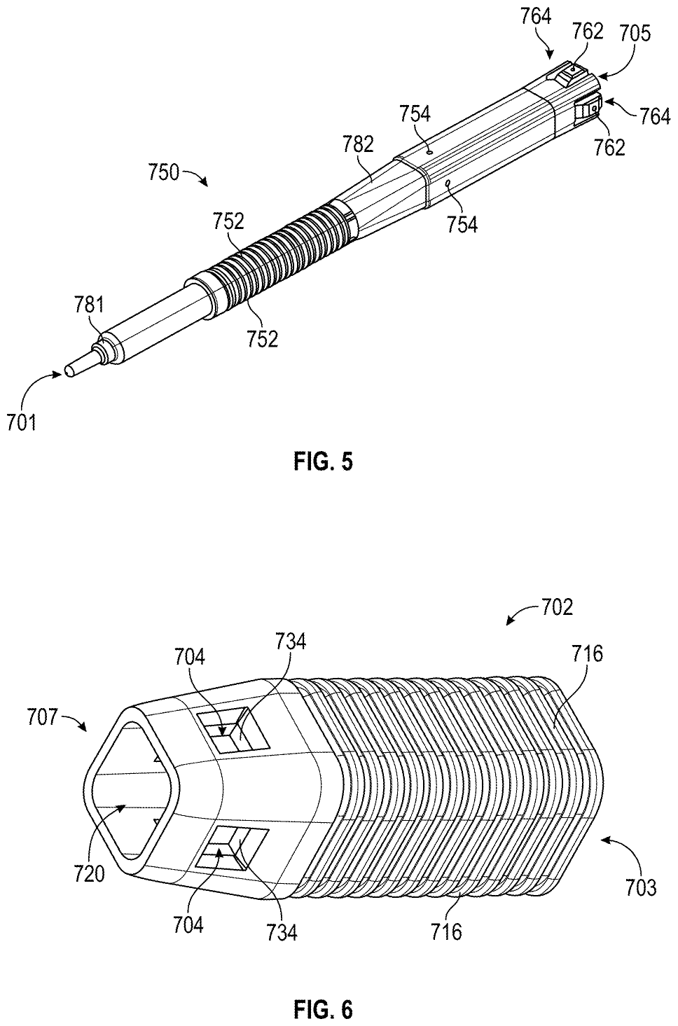

Referring now to FIG. 5, the wand portion 750 is shown in greater detail. As shown, the wand portion 750 includes a plurality of projections 764 (e.g., lateral projections) proximate a second end 705 of the wand portion 750. As will be discussed further herein, the projections 764 may be configured to secure the sample collection device 700 in the extended orientation (e.g., as shown in FIG. 4a). For example, one or more projections 764 may interface with one or more apertures (e.g., apertures 704) in the extension portion 702 to restrict relative movement between the extension portion 702 and the wand portion 750 (e.g., in the extended orientation).

According to various embodiments, the wand portion 750 may include one or more indicators 762 (e.g., visual indicators) located on one or more of the projections 764. The indicators 762 may provide a visual indication to an operator of the sample collection device 700. For example, according to various embodiments, the indicators 762 may be exposed via the one or more apertures (e.g., apertures 704) in the extension portion 702 when the sample collection device 700 is in the extended orientation. According to various embodiments, the indicators 762 may include various shapes and/or colors. For example, the indicator 762 may include coloration of the projection 764 such that the projection 764 has a different appearance (e.g., different color, different shade, etc.) than the shaft 782.

According to various embodiments, the wand portion 750 may include one or more lateral openings 754 located on the shaft 782. According to various embodiments, the openings 754 may serve as an indicator for when the sample collection device 700 is in the collapsed orientation (e.g., as shown in FIG. 4B). Additionally or alternatively, the openings 754 may allow ambient air to fill an internal volume of the wand portion 750 as the sample collection device 700 expands from the collapsed orientation to the extended orientation and/or allow air to escape the internal volume of the wand portion 750 as the sample collection device 700 collapses from the extended orientation to the collapsed orientation, as will be discussed further herein.

According to various embodiments, an extendable sample collection device (e.g., the sample collection device 700) may enable a user to collect samples from areas that may otherwise be difficult to reach, such as the tonsillar pillar and/or the vaginal wall. Further, since the extendable sample collection device may be extended after the sample collection device is removed from the packaging, the footprint of the sample collection device may be reduced, thereby reducing the amount of packaging required to transport the sample collection device. For example, the sample collection device may be pre-packaged within sterile packaging and/or may be configured for one-time use.

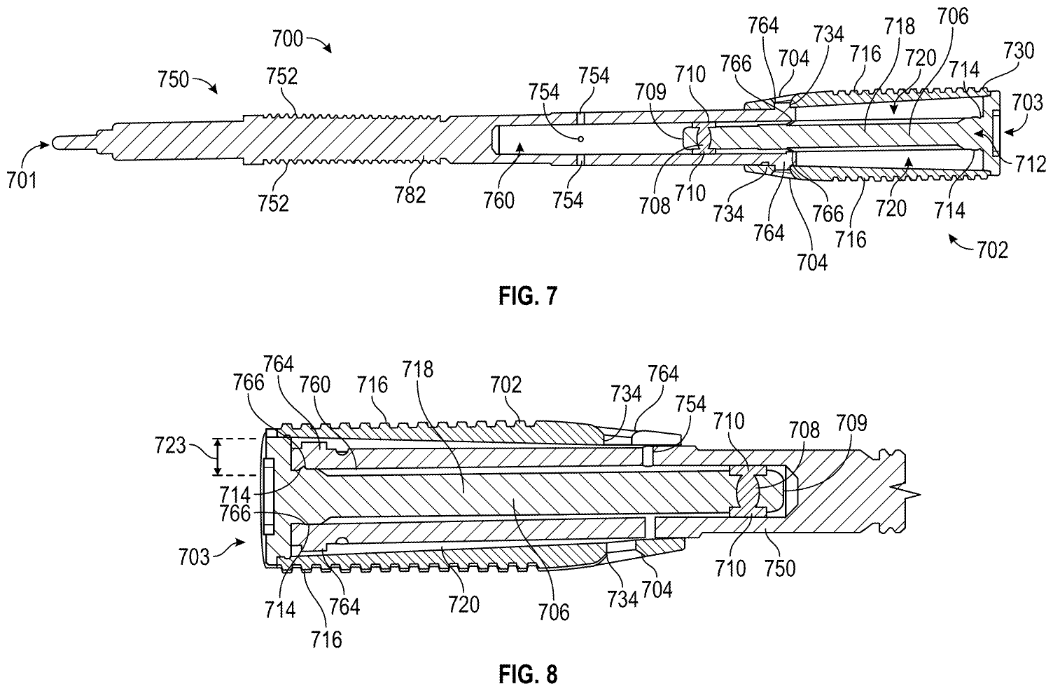

Referring now to FIGS. 7 and 8, a cross sectional view of the sample collection device 700 in the extended orientation and the collapsed orientation, respectively, are shown according to an example embodiment. As shown, the extension portion 702 includes a first opening 720 that extends from a first end 707 of the extension portion 702 and defines an internal volume of the extension portion 702. As shown, the first opening 720 is configured to receive a portion of the wand portion 750 as the sample collection device 700 collapses from an extended position (e.g., as shown in FIG. 7) to a collapsed position (e.g., as shown in FIG. 8).

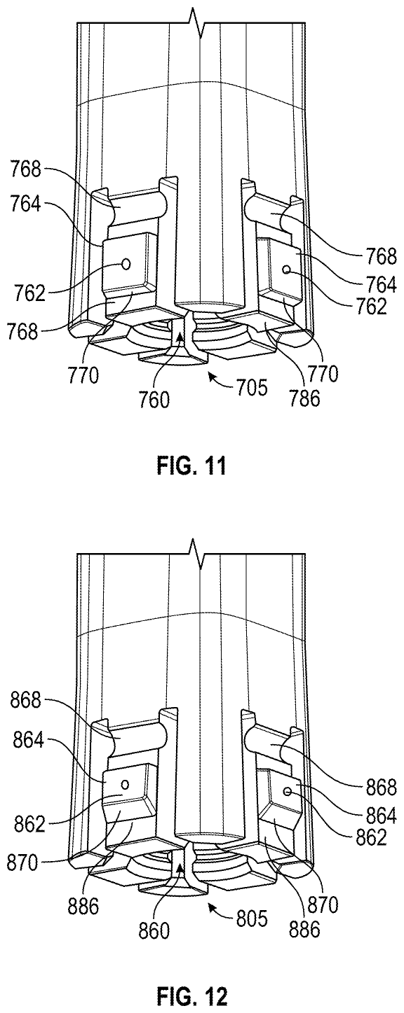

As shown, the projections 764 are received by the apertures 704 while the sample collection device 700 is in the extended orientation (e.g., as shown in FIG. 7). As will be discussed further below with respect to FIGS. 11 and 12, the projections 764 interface with a shoulder 734 of each individual aperture 704 while the sample collection device 700 is in the extended orientation (e.g., as shown in FIG. 7). The interaction between the shoulder 734 and the projection 764 may prevent the sample collection device 700 from being collapsed from the extended orientation to the collapsed orientation. Further, as discussed below, the ring 708 may cause the projections 764 to be biased towards the respective aperture 704, which may further reduce the risk of an incidental collapse of the sample collection device 700.

As shown, the extension portion 702 further includes a locating pin 706 that extends from a second end 703 to a third end 709 and into a second opening 760 in the wand portion 750. As shown, the second opening 760 defines an inner volume of the wand portion 750 that is configured to receive a portion of the locating pin 706 as the sample collection device collapses from an extended position (e.g., as shown in FIG. 7) to a collapsed position (e.g., as shown in FIG. 8). According to various embodiments, the locating pin 706 may provide the sample collection device with additional rigidity. For example, the locating pin 706 may contact the walls of the second opening 760 in response to an off-axis force being applied to the sample collection device 700 to reduce the amount of bending the sample collection device 700 experiences as the off-axis force is applied (e.g., to the tip 701).

As shown, the locating pin 706 includes a ring 708 (e.g., an O-ring) coupled to a shaft 718 of the locating pin 706. Further, the ring 708 includes one or more extensions 710 that extend radially from the ring 708. The extensions 710 may be configured to contact the walls of the second opening 760 as the extension portion 702 translates relative to the wand portion 750. According to various embodiments, the extensions 710 reduce the friction experienced between the locating pin 706 and the walls of the second opening 760 as the extension portion 702 translates relative to the wand portion 750, which may enable smooth sliding action between the extension portion 702 and the wand portion 750. Further, the ring 708 may be compressed within the second opening 760 such that the ring applies a radial force to the inside of the second opening 760, which may cause the projections 764 to be biased towards the respective apertures 704. Applying the radial force to the inside of the second opening 760 may facilitate securing the sample collection device 700 in the extended orientation. According to various embodiments, the ring 708 may be an overmolded elastomer that is coupled to the shaft 718.

As shown, the locating pin 706 further includes an enlarged portion 712 proximate the second end 703. The enlarged portion 712 defines a width that is greater than the width of the shaft 718. As a result, when the enlarged portion is received within the second opening 760, the second end 705 of the wand portion 750 expands (e.g., radially expands) to accommodate the enlarged portion 712.

As shown, the enlarged portion 712 further includes a detent 714 configured to engage an indent 766 of the locating pin 706 while the sample collection device 700 is in the collapsed orientation (e.g., as shown in FIG. 8). According to various embodiments, the interaction between the detent 714 and the indent 766 may restrict movement between the extension portion 702 and the wand portion 750. For example, a predetermined axial force may be required to overcome the resistive force provided by the detent 714 and the indent 766 to extend the sample collection device 700 from the collapsed orientation to the expanded orientation. According to various embodiments, the first opening 720 defines an opening angle 723 such that the second end 705 of the wand portion 750 may expand. For example, the internal diameter of the first opening 720 proximate the second end 705 may be opposite the first end 707 (see FIG. 6).

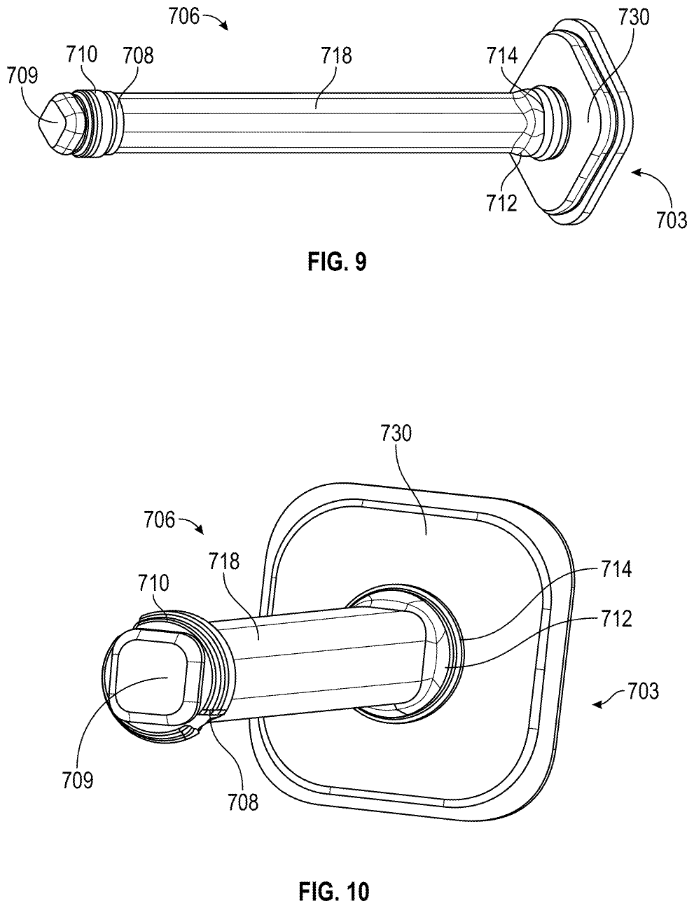

Referring now to FIGS. 9 and 10, perspective views of the locating pin 706 are shown, according to an example embodiment. As shown, the shaft 718 extends from a first surface 730 proximate the second end 703 to a third end 709. Further, the ring 708 is shown to include spaces between the two extensions 710, shown as semi-circular portions of the ring. The spaces between the extensions 710 may allow air to travel past the ring 708 to reduce pressure build up within the first opening 720 and/or the second opening 760 as the extension portion 702 translates relative to the wand portion 750. Further, as discussed above, the openings 754 further reduce pressure build up within the first opening 720 as the extension portion 702 translates relative to the wand portion 750. As shown, the locating pin 706 is a separate component that is coupled to the remainder of the extension portion 702 during assembly of the sample collection device 700. However, according to other various embodiments, the locating pin 706 may be integrally formed with the remainder of the extension portion 702.

Referring now to FIG. 11, the second end 705 of the wand portion 750 is shown, according to an example embodiment. As shown, the wand portion 750 includes a plurality of projections 764 surrounding a circumferential portion of the wand portion 750 proximate the second end 705. As shown, each of the projections 764 includes a flat surface surrounded by a plurality of side walls extending from a tab 786. As shown, the projections 764 are generally rectangular shaped. Further, as shown, an indicator 762 is located on the flat surface of each projection 764. As discussed above, according to various embodiments, the indicators 762 may be exposed via the one or more apertures (e.g., one or more of the apertures 704) in the extension portion 702 when the sample collection device 700 is in the extended orientation.

The tabs 786 are configured to expand to increase a width of the wand portion 750 proximate the second end 705. For example, when the sample collection device 700 is in the collapsed orientation, the enlarged portion 712 of the locating pin 706 may be received within the second opening 760, which may cause the tabs 786 to expand. Further, the tabs 786 include a cut out portion 768 that is less thick that the some or all of the remainder of the tab 786 facilitate expansion of the tabs 786.

As shown, each of the projections 764 include a shoulder 770. The shoulder 770 is configured to engage the shoulder 734 of the aperture 704 (see FIGS. 7 and 8) to prevent the sample collection device 700 from collapsing from the extended orientation to the collapsed orientation. For example, a user of the sample collection device 700 may remove the sample collection device 700 from the respective packaging in the collapsed orientation. The user may then extend the sample collection device 700 into the extended position such that the sample collection device 700 may be exposed to a sample. Exposing the sample collection device 700 may include pressing the tip 701 against a desired surface such that an axial force is applied to the tip 701. According to various embodiments, collapsing of the sample collection device 700 in use may present a risk to the subject the sample is being taken from. Thus, the projections 764 may prevent incidental collapse of the sample collection device 700 during use of the sample collection device.

Referring now to FIG. 12, a second end 805 of a wand portion 850 is shown, according to an alternative example embodiment. The wand portion 850 may share one or more features as the wand portion 750 described above. As shown, the wand portion 850 includes a plurality of projections 864 surrounding a circumferential portion of the wand portion 850 proximate the second end 805. As shown, each of the projections 864 includes a flat surface surrounded by a plurality of side walls extending from a tab 886. As shown, the projections 864 are generally rectangular shaped. Further, as shown, an indicator 862 is located on the flat surface of each projection 864. As discussed above, according to various embodiments, the indicators 862 may be exposed via the one or more apertures (e.g., apertures 704) in the extension portion 702 when the sample collection device 700 is in the extended orientation.

The tabs 886 are configured to expand to increase a width of the wand portion 850 proximate the second end 805. For example, when the sample collection device 800 is in the collapsed orientation, the enlarged portion 812 of the projection 806 may be received within the second opening 860, which may cause the tabs 886 to expand. Further, the tabs 886 include a cut out portion 868 that facilitate expansion of the tabs 886.

As shown, each of the projections 864 includes a shoulder 870. The shoulder 870 is configured to engage the shoulder 734 of the aperture 704 (see FIGS. 7 and 8) to restrict translation between the extension portion 702 and the wand portion 850. For example, as shown, the shoulder 870 defines an angle that interfaces with the shoulder 734 of the aperture 704, as opposed to the square shoulder 770 shown in FIG. 11. The angle of the shoulder 870 is configured to enable the wand portion 850 to collapse within the extension portion 702 in response to a minimum threshold axial force (e.g., a predetermined axial force) being applied to the wand portion 850. The minimum threshold axial force required may depend on the degree of the angle.

According to various embodiments, enabling the sample collection device to collapse under a minimum threshold axial force (e.g., a pre-determined axial force) may reduce the risk of injury to the subject from which the sample is being collected from. Additionally or alternatively, the angled shoulder 870 may enable the sample collection device to collapse when inserted into the cartridge device 300. For example, a user of a sample collection device may remove the sample collection device from the respective packaging in the collapsed orientation. The user may then extend the sample collection device into the extended position such that the sample collection device may be exposed to a sample. After the sample is collected, a user may insert the sample collection device into the cartridge device 300 and apply a predetermined force to the second end of the sample collection device, which may result in a predetermined threshold axial force, which may cause the sample collection device to collapse. According to various embodiments, this may reduce the risk of incidental contact being made with the sample collection device, which may reduce the accuracy of the test, as the test is being completed.

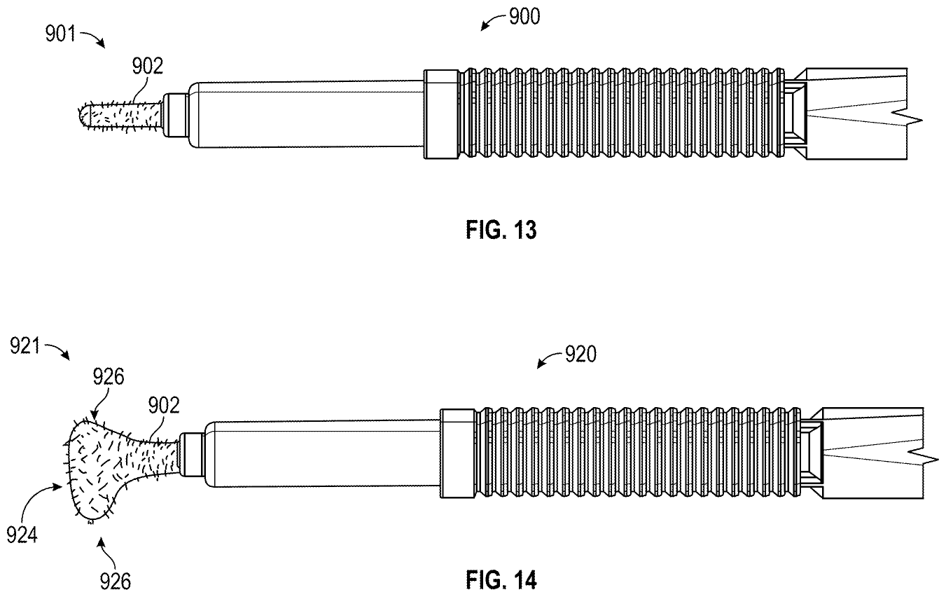

Referring now to FIG. 13, a side view of a portion of a sample collection device 900 including an example tip 901 is shown, according to an example embodiment. It should be appreciated that the tip 901 may be included on any of the sample collection devices described herein (e.g., the sample collection device 200, the sample collection device 700, etc.). The tip 901 is configured to be exposed to a sample such that a volume of sample may be collected. The tip 901 may be include a rigid plastic material. For example, the tip 901 may be formed from an acrylonitrile butadiene styrene (ABS) polymer. According to various embodiments, the tip 901 is at least partially injection molded.

As shown, the tip 901 is covered in a layer of material 902. The layer of material 902 be a soft and/or absorbent material. For example, the layer of material 902 may include a layer of flocking that is applied to the tip 901. The flocking may be a polyester bicomponent fiber that is attached to the tip 901 with an adhesive. According to various embodiments, the flocking may be Puritan™ PurFlock™. According to various embodiments, the flocking may provide an absorbent layer to collect and retain the target sample. According to various embodiments, the flocking may function to cushion the tip 901 and reduce irritation or damage to sensitive tissues during use of the sample collection device.

Referring now to FIG. 14, a side view of a portion of a sample collection device 920 including another example tip 921 is shown, according to an example embodiment. It should be appreciated that the tip 921 may be included on any of the sample collection devices described herein (e.g., the sample collection device 200, the sample collection device 700, etc.). The tip 921 is configured to be exposed to a sample such that a volume of sample may be collected. The tip 921 may include a rigid plastic material. For example, the tip 921 may be formed from an ABS polymer. According to various embodiments, the tip 921 is at least partially injection molded.

As shown, the tip includes an enlarged area 924 proximate the end of the tip 921. According to various embodiments, the enlarged area 924 provides additional surface area to contact the desired area to collect a sample, which may reduce to potential for damage to the surrounding tissue while collecting the sample. According to various embodiments, the enlarged portion includes lateral sides 926 surrounding a circumferential portion of the tip 921. As shown, the lateral sides 926 are not parallel with one another, but rather define a convex surface surrounding the tip 921. According to various embodiments, the enlarged portion is generally egg-shaped. According to various embodiments, the end of the tip 921 includes a relatively large and flat surface to increase the surface area of the end of the tip 921.

As shown, the tip 921 is covered in a layer of material 922. The layer of material 922 may be a soft and/or absorbent material. For example, the layer of material 922 may include a layer of flocking that is applied to the tip 921. The flocking may include a polyester bicomponent fiber that is attached to the tip 921 (e.g., via an adhesive). According to various embodiments, the flocking may be Puritan™ PurFlock™. According to various embodiments, the flocking may provide an absorbent layer to collect and retain the target sample. According to various embodiments, the flocking may function to cushion the tip 921 and reduce irritation or damage to sensitive tissues during use of the sample collection device.

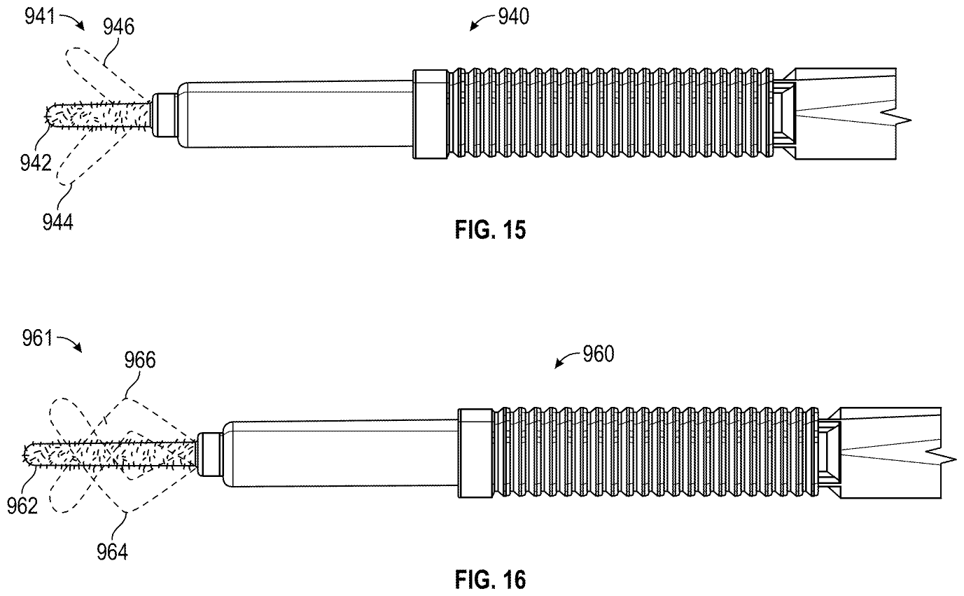

Referring now to FIG. 15, a side view of a portion of a sample collection device 940 including yet another example tip 941 is shown, according to an example embodiment. It should be appreciated that the tip 941 may be included on any of the sample collection devices described herein (e.g., the sample collection device 200, the sample collection device 700, etc.). The tip 941 is configured to be exposed to a sample such that a volume of sample may be collected. The tip 941 may include a flexible plastic material. For example, the tip 941 may be formed from a thermoplastic elastomer. According to various embodiments, the tip 941 is injection molded. According to other embodiments, the tip 941 may include a flexible material (e.g., a flexible elastomeric material) that is not covered in flocking. For example, the entire tip 941 may be manufactured from a flexible material such as an elastomeric material.

According to various embodiments, the flexible tip 941 enables the tip 941 to conform to various contours as need to collect a sample from various locations. For example, the tip 941 may be configured to bend between at least a first position 944 and a second position 946. According to various embodiments, the flexible tip 941 may reduce the risk of irritation or damage to sensitive tissues during use of the sample collection device (e.g., by conforming to the contours of the sample collection location). The flexible tip 941 may be configured to flex more proximate one or more portions of the flexible tip 941. For example, as shown in FIG. 15, the flexible tip may be 941 configured to flex about an inflection point proximate the wand body. In another example, as shown in FIG. 16, the flexible tip 941 may be configured to flex about a first inflection point proximate the wand body and a second inflection point at a midpoint between the ends of flexible tip 941.

As shown, the tip 941 is covered in a layer of material 942. The layer of material 942 be a soft and/or absorbent material. For example, the layer of material 942 may include a layer of flocking that is applied to the tip 941. The flocking may be a polyester bicomponent fiber that is attached to the tip 941 (e.g., via an adhesive). For example, a layer of substrate may be coupled to the tip 941 such that the flocking may be coupled to the tip 941. According to various embodiments, the flocking may be Puritan™ PurFlock™ According to various embodiments, the flocking may provide an absorbent layer to collect and retain the target sample. According to various embodiments, the flocking may function to cushion the tip 941 and reduce irritation or damage to sensitive tissues during use of the sample collection device.

Referring now to FIG. 16, a side view of a portion of a sample collection device 960 including still another example tip 961 is shown, according to an example embodiment. It should be appreciated that the tip 961 may be included on any of the sample collection devices described herein (e.g., the sample collection device 200, the sample collection device 700, etc.). The tip 961 is configured to be exposed to a sample such that a volume of sample may be collected. The tip 961 may include a flexible plastic material. For example, the tip 961 may be formed from a thermoplastic elastomer. According to various embodiments, the tip 961 is injection molded.

According to various embodiments, the flexible tip 961 enables the tip 961 to include more than one curved portion, allowing the flexible tip 961 to conform to various contours as need to collect a sample from various locations. For example, the tip 961 may be configured to bend between at least a first position 964 and a second position 966. According to various embodiments, the flexible tip 961 may reduce the risk of irritation or damage to sensitive tissues during use of the sample collection device.

As shown, the tip 961 is covered in a layer of material 962. The layer of material 962 be a soft and/or absorbent material. For example, the layer of material 962 may include a layer of flocking that is applied to the tip 961. The flocking may be a polyester bicomponent fiber that is attached to the tip 961 (e.g., via an adhesive). According to various embodiments, the flocking may be Puritan™ PurFlock™. According to various embodiments, the flocking may provide an absorbent layer to collect and retain the target sample. According to various embodiments, the flocking may function to cushion the tip 961 and reduce irritation or damage to sensitive tissues during use of the sample collection device.

Unless otherwise defined, each technical or scientific term used herein has the same meaning as commonly understood by one of ordinary skill in the art to which this disclosure belongs. In accordance with the claims that follow and the disclosure provided herein, the following terms are defined with the following meanings, unless explicitly stated otherwise.

The term “about” or “approximately,” when used before a numerical designation or range (e.g., pressure or dimensions), indicates approximations which may vary by (+) or (−) 5%, 1% or 0.1%.

As used in the specification and claims, the singular form “a”, “an” and “the” include both singular and plural references unless the context clearly dictates otherwise. For example, the term “a molecule” may include, and is contemplated to include, a plurality of molecules. At times, the claims and disclosure may include terms such as “a plurality,” “one or more,” or “at least one;” however, the absence of such terms is not intended to mean, and should not be interpreted to mean, that a plurality is not conceived.

As used in the specification and claims, “at least one of” means including, but not limited to, one or more of any combination of the following. For example, “at least one of A, B, and C” or “at least one of A, B, or C” means including, but not limited to, A(s) or B(s) or C(s) or A(s) and B(s) or A(s) and C(s) or B(s) and C(s) or A(s) and B(s) and C(s); none of which excludes other elements such as D(s), E(s), etc.

As used herein, the term “comprising” or “comprises” is intended to mean that the devices, systems, and methods include the recited elements, and may additionally include any other elements. “Consisting essentially of” shall mean that the devices, systems, and methods include the recited elements and exclude other elements of essential significance to the combination for the stated purpose. Thus, a device or method consisting essentially of the elements as defined herein would not exclude other materials or steps that do not materially affect the basic and novel characteristic(s) of the claimed invention. “Consisting of” shall mean that the devices, systems, and methods include the recited elements and exclude anything more than a trivial or inconsequential element or step. Embodiments defined by each of these transitional terms are within the scope of this disclosure.

Although the foregoing has included detailed descriptions of some embodiments by way of illustration and example, it will be readily apparent to those of ordinary skill in the art in light of the teachings of these embodiments that numerous changes and modifications may be made without departing from the spirit or scope of the appended claims.

1. A sample collection device for collecting a biological sample for analysis in a detection system, comprising:
a body including a wand portion; and
a tip extending from a proximal end of the wand portion and configured to receive a sample, the tip including a tip body comprising a flexible material.

2. The sample collection device of claim 1, wherein the tip further includes a layer of flocking coupled to the tip body.

3. The sample collection device of claim 1, wherein the tip includes a transparent portion configured to expose an inner volume of the tip.

4. The sample collection device of claim 1, wherein the tip includes an enlarged portion that defines convex lateral sides of the tip.

5. The sample collection device of claim 1, wherein the flexible material includes an elastomeric material.

6. The sample collection device of claim 2, wherein the flocking includes a polyester bicomponent fiber.

7. The sample collection device of claim 1, wherein the body extends along an axis and the tip is flexible about the axis.

8. The sample collection device of claim 1, wherein the tip defines a first diameter at a first end proximate the wand portion and a second diameter at a second end opposite the first end, the second diameter being larger than the first diameter.

9. The sample collection device of claim 2, wherein the flocking is coupled to the tip via adhesive.

10. The sample collection device of claim 2, wherein the flocking is configured to absorb the biological sample.

11. The sample collection device of claim 1, wherein the tip body is configured to flex proximate a first inflection point proximate the wand portion.

12. The sample collection device of claim 11, wherein the tip body is configured to flex proximate a second inflection point proximate between a first end of the tip and a second end of the tip.

13. A sample collection device for collecting a biological sample for analysis in a detection system, comprising a tip extending along an axis, configured to receive a sample, the tip including a tip body and a layer of flocking coupled to the tip body.

14. The sample collection device of claim 13, wherein the tip includes an enlarged portion that defines convex lateral sides of the tip.

15. The sample collection device of claim 13, wherein the tip body includes an elastomeric material.

16. The sample collection device of claim 13, wherein the flocking includes a polyester bicomponent fiber.

17. The sample collection device of claim 13, wherein the flocking is coupled to the tip body via an adhesive.

18. The sample collection device of claim 13, further comprising a wand portion coupled to the tip, wherein the tip defines a first diameter at a first end proximate the wand portion and a second diameter at a second end opposite the first end, the second diameter being larger than the first diameter.
